(19) United States
(12) Patent Application Publication
(10) Pub. No.: US 2011/0311607 A1
(43) Pub. Date: Dec. 22, 2011

(54) PUNCTAL PLUGS WITH CONTINUOUS OR PULSATILE DRUG RELEASE MECHANISM

(76) Inventors: Bret A. Coldren, Vista, CA (US); Günter Solms, Jacksonville Beach, FL (US); Gary Yewey, Jacksonville, FL (US); Victor Lust, Jacksonville, FL (US); Jeffrey Roffman, St. Johns, FL (US)

(21) Appl. No.: 13/155,498

(22) Filed: Jun. 8, 2011

Related U.S. Application Data
(60) Provisional application No. 61/356,134, filed on Jun. 18, 2010.

(51) Int. Cl. A61F 2/14; A61P 27/04

(52) U.S. Cl. 424/427

(57) ABSTRACT

Disclosed are lacrimal inserts and their method of use for delivery of medication to the eye. The plug includes a body portion sized to pass through a lacrimal punctum and be positioned within a lacrimal canaliculus of the eyelid. The plug may contain a core, or reservoir, at least partially within the body portion comprising a therapeutic agent that is configured to controlled release into the eye.

### CROSS REFERENCE TO RELATED APPLICATIONS

This application relates to U.S. patent application Ser. No. 61/356,134, filed Jun. 18, 2010; all applications are herein incorporated by reference in their entireties.

### FIELD OF THE INVENTION

This invention relates to an ophthalmic insert and method for the release of medication to the eye for the treatment of eye disorders. More specifically, the invention relates to punctal plugs sized to pass through a lacrimal punctum and be positioned within a lacrimal canaliculus of the eyelid and containing medication for controlled release into the eye in a therapeutically effective amount in a pulsatile or continuous manner, or combinations thereof

### BACKGROUND OF THE INVENTION

Active agents frequently are administered to the eye for the treatment of ocular diseases and disorders. Conventional means for delivering active agents to the eye involve topical application to the surface of the eye. The eye is uniquely suited to topical administration because, when properly constituted, topically applied active agents can penetrate through the cornea and rise to therapeutic concentration levels inside the eye. Active agents for ocular diseases and disorders may be administered orally or by injection, but such administration routes are disadvantageous in that, in oral administration, the active agent may reach the eye in too low a concentration to have the desired pharmacological effect and their use is complicated by significant, systemic side effects and injections pose the risk of infection.

The majority of ocular active agents are currently delivered topically using eye drops which, though effective for some applications, are inefficient. When a drop of liquid is added to the eye, it overfills the conjunctival sac, the pocket between the eye and the lids, causing a substantial portion of the drop to be lost due to overflow of the lid margin onto the cheek. In addition, a substantial portion of the drop that remains on the ocular surface is drained into the lacrimal puncta, diluting the concentration of the drug.

To compound the problems described above, patients often do not use their eye drops as prescribed. Often, this poor compliance is due to an initial stinging or burning sensation caused by the eye drop. Certainly, instilling eye drops in one's own eye can be difficult, in part because of the normal reflex to protect the eye. Therefore, sometimes one or more drops miss the eye. Older patients may have additional problems instilling drops due to arthritis, unsteadiness, and decreased vision, and pediatric and psychiatric patient populations pose difficulties as well.

It is known to use devices that may be inserted into one or more of an orifice of an individual's eye, such as a lacrimal punctum, to deliver active agents. One disadvantage of using such devices to deliver agents is that much of the agent may delivered in an initial, large bolus upon insertion of the device into the eye rather than a more linear delivery of the agent over time.

Prior topical sustained release systems include gradual release formulations, either in solution or ointment form, which are applied to the eye in the same manner as eye drops but less frequently. Such formulations are disclosed, for example, in U.S. Pat. No. 3,826,258 issued to Abraham and U.S. Pat. No. 4,923,699 issued to Kaufman. Due to their method of application, however, these formulations result in many of the same problems detailed above for conventional eye drops. In the case of ointment preparations, additional problems are encountered such as a blurring effect on vision and the discomfort of the sticky sensation caused by the thick ointment base.

Alternatively, sustained release systems have been configured to be placed into the conjunctival cul-de-sac, between the lower lid and the eye. Such units typically contain a core drug-containing reservoir surrounded by a hydrophobic copolymer membrane which controls the diffusion of the drug. Examples of such devices are disclosed in U.S. Pat. No. 3,618,604 issued to Ness, U.S. Pat. No. 3,626,940 issued to Zaffaroni, U.S. Pat. No. 3,845,770 issued to Theeuwes et al., U.S. Pat. No. 3,962,414 issued to Michaels, U.S. Pat. No. 3,993,071 issued to Higuchi et al., and U.S. Pat. No. 4,014,335 issued to Arnold. However, due to their positioning, the units are uncomfortable and poor patient acceptance is again encountered.

### BRIEF DESCRIPTION OF THE DRAWINGS

FIG. 1 shows a cross-sectional view of a lacrimal device according to an illustrative embodiment of the invention having an interior surface configured to include a plurality of stiction elements.

FIG. 1A shows a cross-sectional view of a lacrimal device according to an illustrative embodiment of the invention having an interior surface configured to include a plurality of stiction elements, and an activation element.

FIG. 2 shows a cross-sectional view of a lacrimal device according to another illustrative embodiment of the invention where the tube wall is configured without stiction elements.

FIG. 2A shows a cross-sectional view of a lacrimal device according to another illustrative embodiment of the invention where the tube wall is configured without stiction elements, and an activation element.

FIG. 3 is a partial depiction another embodiment of the invention, in cross-section, showing a terminal valve and a restriction element proximate thereto.

FIG. 4 depicts a device according to FIG. 3 actuated to deliver a quantity of active agent formulation.

FIG. 5 shows another illustrative embodiment of a lacrimal insert according to the present invention in cross-section.

FIG. 5A shows another illustrative embodiment of a lacrimal insert according to the present invention in cross-section, and an activation element.

FIG. 6 shows a cross-sectional view of the structure of an exemplary embodiment of a microcapsule according to the present invention.

FIG. 7 shows a cross-sectional view according to another illustrative embodiment of the invention of a tubular lacrimal device.

FIG. 7A shows a cross-sectional view according to another illustrative embodiment of the invention of a tubular lacrimal device, and an activation element.

FIG. 8A shows a partial, cross-sectional view of an exemplary, tubular, lacrimal device having a terminal valve.

FIG. 8B illustrates the device of FIG. 8A where the valve is actuated to permit the release of material therethough.

FIG. 9 illustrates one possible profile for pressure change in the lacrimal device versus release-rate of material through the terminal valve.

FIG. 10 illustrates one possible profile for the release rate of material through the terminal valve as a function of time and the extent to which the valve is open relative to its maximum.

FIG. 11 depicts another illustrative embodiment of a tubular lacrimal insert, in cross-section, and a metering valve disposed therein.

FIG. 11A depicts another illustrative embodiment of a tubular lacrimal insert, in cross-section, and a metering valve disposed therein, and an activation element.

FIG. 12 illustrates another exemplary embodiment of the present invention in which a tubular lacrimal insert having an alternating series of barrier layers and active-agent containing layers.

FIG. 12A illustrates another exemplary embodiment of the present invention in which a tubular lacrimal insert having an alternating series of barrier layers and active-agent containing layers, and an activation element.

FIG. 13 illustrates another exemplary embodiment of the present invention in which a tubular lacrimal insert includes a piston and a metering valve element.

FIG. 13A illustrates another exemplary embodiment of the present invention in which a tubular lacrimal insert includes a piston and a metering valve element, and an activation element.

FIG. 14 illustrates another exemplary embodiment of the present invention, showing a partial cross-section of an alternative structure for a terminal valve element.

FIG. 15 illustrates another exemplary embodiment of the present invention, showing a partial cross-section of an alternative structure for a terminal valve element.

FIG. 16 illustrates another exemplary embodiment of the present invention, showing a partial cross-section of an alternative structure for a terminal valve element.

FIG. 17 illustrates another exemplary embodiment of the present invention, showing a partial cross-section of an alternative structure for a terminal valve element.

FIG. 18 illustrates another exemplary embodiment of the present invention, showing a partial cross-section of an alternative structure for a terminal valve element.

FIG. 19 illustrates another exemplary embodiment of the present invention, showing a partial cross-section of an alternative structure for a terminal valve element.

FIG. 20 illustrates another exemplary embodiment of a tubular lacrimal insert according to the present invention.

FIG. 20A illustrates another exemplary embodiment of a tubular lacrimal insert according to the present invention, and an activation element.

### DETAILED DESCRIPTION OF ILLUSTRATIVE EMBODIMENTS OF THE INVENTION

Punctal plugs have been in use for decades now to treat conditions of dry eye. More recently they have gained attention for use as drug delivery systems for the treatment of ocular diseases and conditions. Several challenges exist with formulating a drug to release at the desired daily rate and or dose that will give efficacy while limiting adverse events.

Diffusion based drug delivery systems are characterized by release rate of drug is dependent on its diffusion through inert water insoluble membrane barrier. There are basically diffusion designs: Reservoir devices and matrix devices. Reservoir devices are those in which a core of drug is surrounded by polymeric membrane. The nature of membrane determines the rate of release of drug from system. The process of diffusion is generally described by a series of equations governed by Fick's first law of diffusion. A matrix device consists of drug dispersed homogenously throughout a polymer.

Reservoir and matrix drug delivery systems are considered diffusion based sustained release systems and constitute any dosage form that provides medication over an extended period of time. The goal of a sustained release system is to maintain therapeutic levels of drug for an extended period and this is usually accomplished by attempting to obtain zero-order release from the sustained release system. Sustained release systems generally do not attain this type of release profile but try to approximate it by releasing in a slow first order manner. Over time, the drug release rate from reservoir and matrix sustained release systems will decay and become non therapeutic.

Zero-order drug release constitutes drug release from a drug delivery system at a steady sustained drug release rate, that is, the amount of drug that is released from the drug delivery system over equal time intervals does not decay and remains at the therapeutic level. This “steady sustained release drug delivery system” is referred to as a zero-order drug delivery system and has the potential to provide actual therapeutic control by its controlled release.

Another drug release profile is referred to as pulsatile drug delivery. Pulsatile drug delivery is intended to release a therapeutic amount of a therapeutic agent at regular intervals. Turning now to the drawing figures, which are meant to be instructive, but not exhaustive of the possible structure and materials of the embodiments of the present invention and wherein similar reference numerals refer to similar structure.

As used herein, the term “active agent” refers to an agent capable of treating, inhibiting, or preventing a disorder or a disease. Exemplary active agents include, without limitation, pharmaceuticals and nutraceuticals. Preferred active agents are capable of treating, inhibiting, or preventing a disorder or a disease of one or more of the eye, nose and throat.

As used herein, the term “punctal plug” refers to a device of a size and shape suitable for insertion into the inferior or superior lacrimal canaliculus of the eye through, respectively, the inferior or superior lacrimal punctum. Exemplary and illustrative devices are disclosed in U.S. Pat. No. 6,196,993 and U.S. Published Patent Application No. 20090306608A1, both of which are hereby incorporated by reference in their entireties. Examples of punctual plugs with osmotically controlled drug delivery systems are also described in commonly owned, copending U.S. Application Ser. No. 61/322127, filed on Apr. 8, 2010, which is hereby incorporated by reference in its entirety.

As used herein, the term “opening” refers to an opening in the body of a device of the invention of a size and shape through which the active agent can pass. Preferably, only the active agent and formulation can pass through the opening. The opening may be covered with a membrane, single or multiple pores, mesh, grid or it may be uncovered. The membrane, mesh, or grid may be one or more of porous, semi-porous, permeable, semi-permeable, and biodegradable.

The devices of the invention have a reservoir in which is found an active agent-containing material and an active agent therein. The active agent may be dispersed throughout the active agent-containing material or dissolved within the material. Alternatively, the active agent may be contained in inclusions, particulates, droplets, beads, or micro-encapsulated within the material. Still as another alternative, the active agent may be covalently bonded to the material and released by hydrolysis, enzymatic degradation and the like. Yet as another alternative, the active agent may be in a reservoir within the material.

It is a discovery of the invention that the active agent may be released in a controlled manner, meaning over a period of time by using an active agent-containing material in which the agent is present in a substantially continuous concentration gradient throughout the material or by using a discontinuous concentration gradient. This is in contrast to a device that exhibits a “burst” or immediate release upon insertion of an amount of active agent that is greater than the average release rate over time.

The local gradient may be controlled by placing more active agent at one location in the active agent-containing material relative to another location. For example, the concentration profile can be a continuous gradient from one end of the material to the other. Alternatively, the matrix may be have a discontinuous gradient, meaning that one section of the material has a first concentration and the concentration abruptly changes to a second, different concentration in an adjacent section of the matrix. The diffusivity for the active agent may also be spatially controlled by varying one or more of the chemical composition, porosity, and crystallinity of the active agent-containing material.

Additionally, the spatial variation of the material's cross-sectional geometry may be used to control diffusivity. For example, if the material was in the form of a straight rod that has a uniform active agent concentration, diffusivity will be reduced when the area at the open end of the material is significantly smaller than the average of the entire material. Preferably, the material area at the open end of the device is no more than one-half of the average cross sectional area of the material, meaning the cross section determined perpendicular to the primary dimension of active agent transport use.

One of ordinary skill in the art will recognize that, depending on how one varies one or more of the local concentration gradients, the diffusivity of the active agent from the material, and the spatial variation of the cross-sectional geometry of the device, a variety of release profiles may be obtained including, without limitation first order, second order, biphasic, pulsatile and the like. For example, either or both of the active agent concentration and diffusivity may increase from the surface to the center of the active agent-containing material in order to achieve more initial release. Alternatively, either or both may be increased or decreased and then increased again within the material to achieve a pulsatile release profile. The ability to achieve a variety of release profiles by varying local concentration gradient, the diffusivity of the active agent, and the spatial variation of the cross-sectional geometry may eliminate the need for rate-limiting membranes in the device.

Alternatively, it is a discovery of the invention that small “bursts” of active-agent containing material may produce therapeutically effective dosaging of active-agent into the desired treatment region. Such bursts may be accomplished by the periodic introduction of encapsulated active agent, as might be found in microcapsules, microbeads, etc, or by creating a reservoir of active-agent containing material that delivers a period bolus of therapeutic material (i.e., active agent) by mechanical, electrical, chemical, or other means that are determined by the structure and geometry of a reservoir within the lacrimal insert.

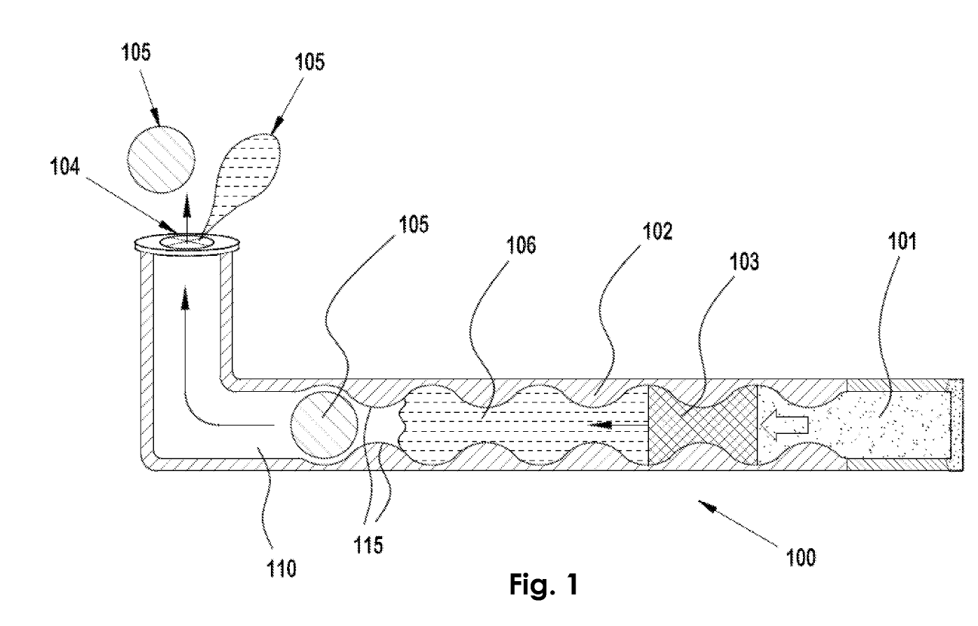

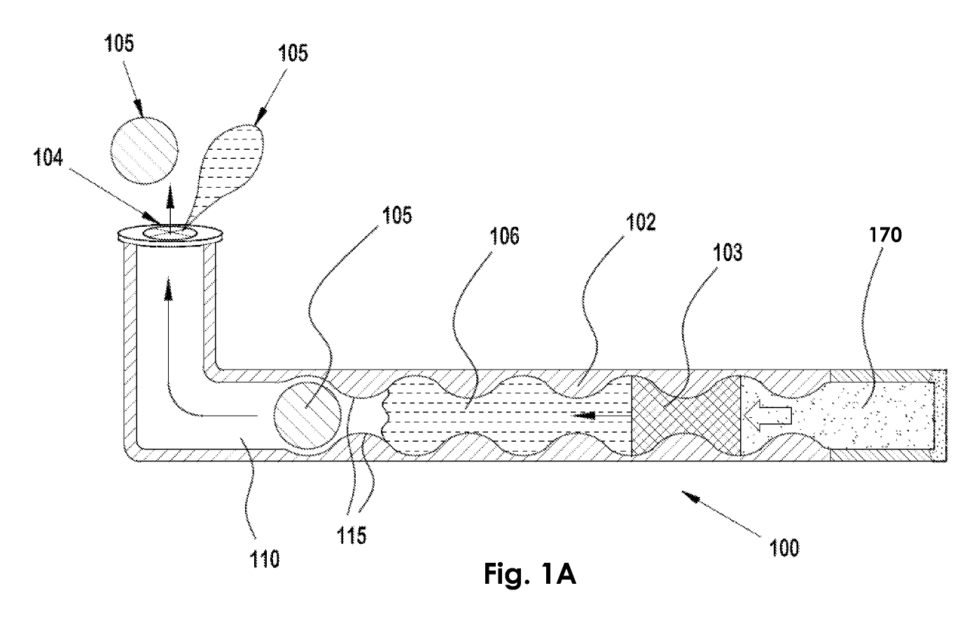

For example, as illustrated in FIG. 1, the invention may be characterized as an osmotic or swellable hydrogel engine with a series of intermittent stops or stiction elements, combined with a drug reservoir and terminal orifice or valving element, to result in a pulsatile release rate of therapeutic substance(s) to the eye. The embodiment of the invention shown in FIG. 1 may comprise a tubular lacrimal insert 100 from about 1 mm to about 10 mm in length, and from about 0.2 mm to about 2 mm in diameter. The lacrimal insert 100 may include a cavity 110 defined by active-agent impermeable inner surface walls 115. As shown in the instant embodiment, the inner surface walls 115 may include protrusions, or stiction features 102, that may be, but not limited to, hemispherical as shown, and a piston 103 whose rate of travel through the reservoir 110 is regulated by an osmotic pump formulation 101 that exerts pressure against the piston 103 as it expands. Typically, the expansion of the osmotic pump formulation 101 may be caused by the interaction of the material that comprises the osmotic pump formulation 101 and lacrimal fluid (or, more precisely in some embodiments, the water contained therein). While the piston and tube shapes should be complementary to each other in order to seal, they need not be round. Square, triangular, trapezoidal, etc cross-sections may be used, and the internal and external profile of the tube need not be the same. The device may be configured for the pulsatile (or oscillatory) release of active agent over a period of 1 day to 1 year. The active agent 106 may be comprised of a fluid or semisolid formulation. Alternatively, the active agent 106 may comprise a microcapsule or microsphere 105. The active-agent-impermeable tubular body inner surface walls 115 contains stiction elements 102 that interact with a spherical, hourglass or other shaped piston 103 in a complementary manner. Depending on the desired release profile, the stiction elements 102 may be evenly spaced, as shown in FIG. 1, or unevenly spaced (not shown) to control the movement of the piston 103. In an alternate embodiment illustrated in FIG. 1A, an externally activated pumping mechanism 170 is used to propel the piston 103. The pumping mechanism 170 may be activated by an electromagnetic or radio frequency pulse or signal, magnetic, piezoelectric, electrostatic or similar means.

Controlled water diffusion into the osmotic pump 101 generates pressure on the piston which by virtue of the stiction elements 102, leads to motion of the piston 103 and, subsequently, substantially periodic emission of the active agent formulation 106 or active agent containing microspheres/capsules 105. The tubular lacrimal device 100 may also comprise a terminal valve element 104 to accentuate the pulsing action and/or minimize diffusion of active agent out of the device during the time period between pulses, which may range from 1 hour to 1 month. The total number of contained stiction elements 102, and hence pulses (or boluses) of material released through the terminal valve 104, depends upon the specific active agent dose requirements of the application, generally at least 2 to 300 stiction elements 103.

The invention may, further, be broadly characterized as an osmotic or swellable hydrogel engine, with or without intermittent stops, that drives a drug load in the form of discrete particles (spheroids or cylinders) combined with a terminal orifice or valving element, which are emitted in a pulsatile pattern to the eye. Discrete particles can be water soluble or insoluble. Said discrete particles can be solid or hollow, compressible or friable, and emitted either intact from the device or in a fragmented or solvated state. Also, the drug load may comprise multiple and/or alternating populations of discrete particles that comprise two or more different drugs. The discrete particles may optionally be surrounded by a water-protective agent such as hydrophobic oils or polymers.

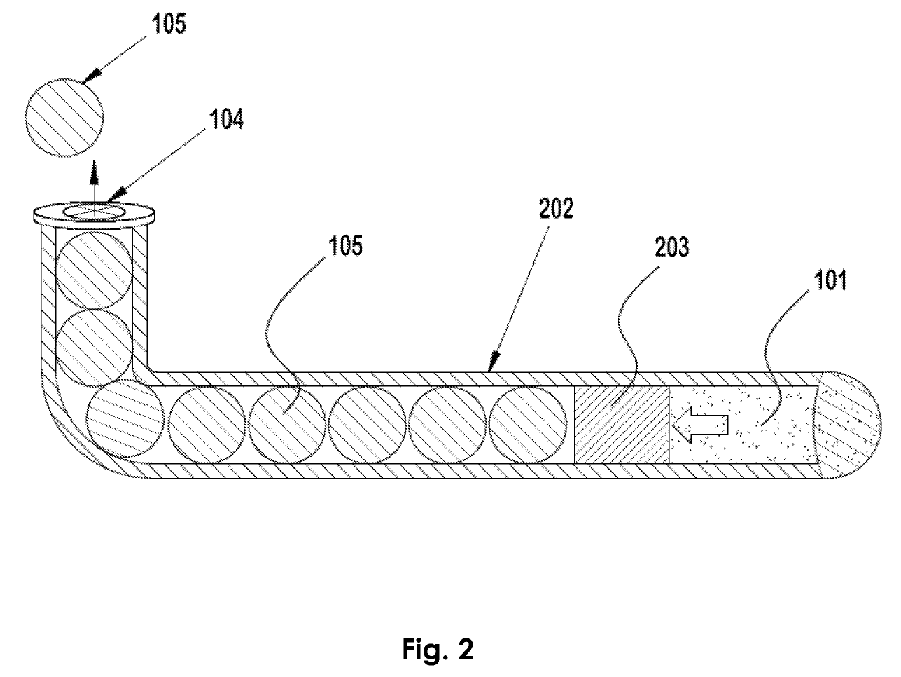

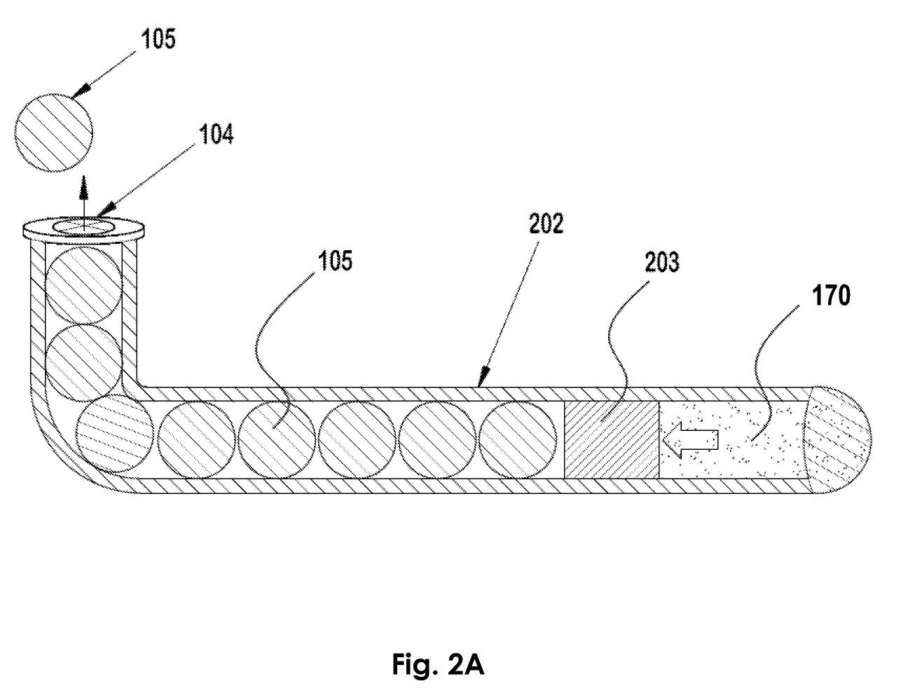

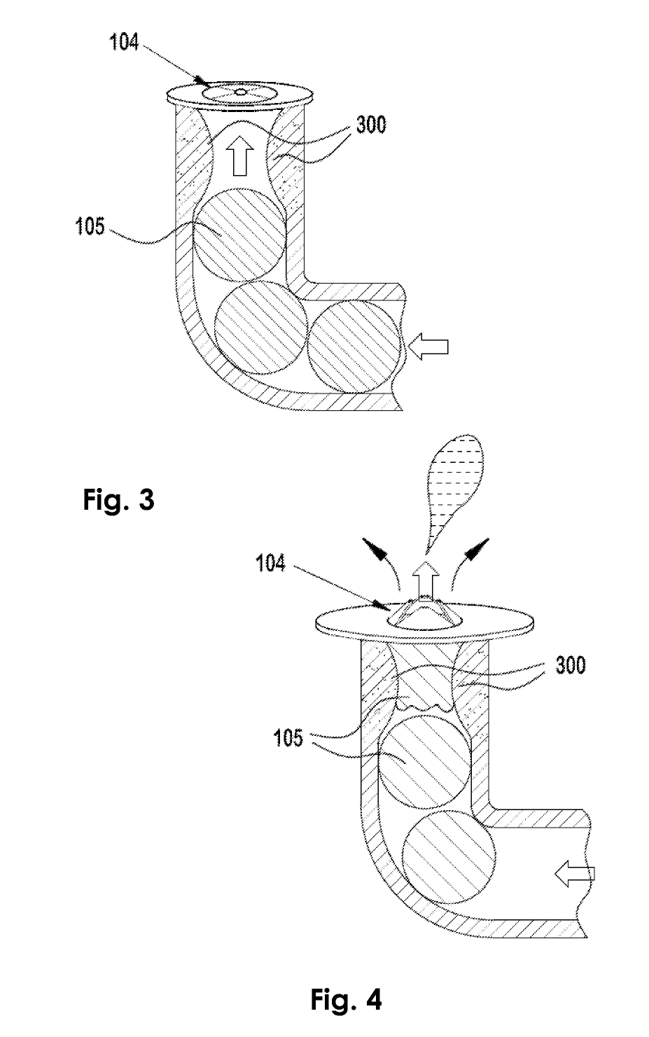

FIG. 2 illustrates another exemplary embodiment according to the present invention in which the tubular lacrimal device 100 has relatively smooth inner surface walls 115 defining the reservoir 110. The inner surface walls 115 lack stiction elements 102 in the tube wall 202, relying solely on the osmotic pump 101 and piston 203 to drive the periodic emission of soluble active agent containing microspheres 105 from the terminal valve element 104. Further, as shown illustratively in FIG. 3, the terminal end of the tubular lacrimal device 100 may comprise a terminal valve 104 in combination with one or more cooperative restriction elements 300. In this configuration, the osmotic pump 101 forces the movement of microspheres 105 through reservoir 110 of the device 100. As the microspheres pass through the restriction element 300, the restriction on the spheres 105 caused by the decreasing size of the cavity through which they pass causes the microspheres 105 to burst and the active-agent containing material included therein is emitted via the terminal valve 104, as shown in FIG. 4. In an alternate embodiment illustrated in FIG. 2A, an externally activated pumping mechanism 170 is used to propel the piston 103. The pumping mechanism 170 may be activated by an electromagnetic or radio frequency pulse or signal, magnetic, piezoelectric, electrostatic or similar means.

The osmotic or swellable hydrogel engine described heretofore and hereinafter, with or without intermittent stops, that drives a drug load which is dispersed between discrete inert particles (spheroids or cylinders), combined with a terminal orifice or valving element, may result in a pulsatile drug delivery pattern of one or more drugs to the target site, such as the eye. The discrete particles can be water soluble or insoluble. They may also be solid or hollow, compressible or friable, and emitted either intact from the device or in a fragmented or solvated state

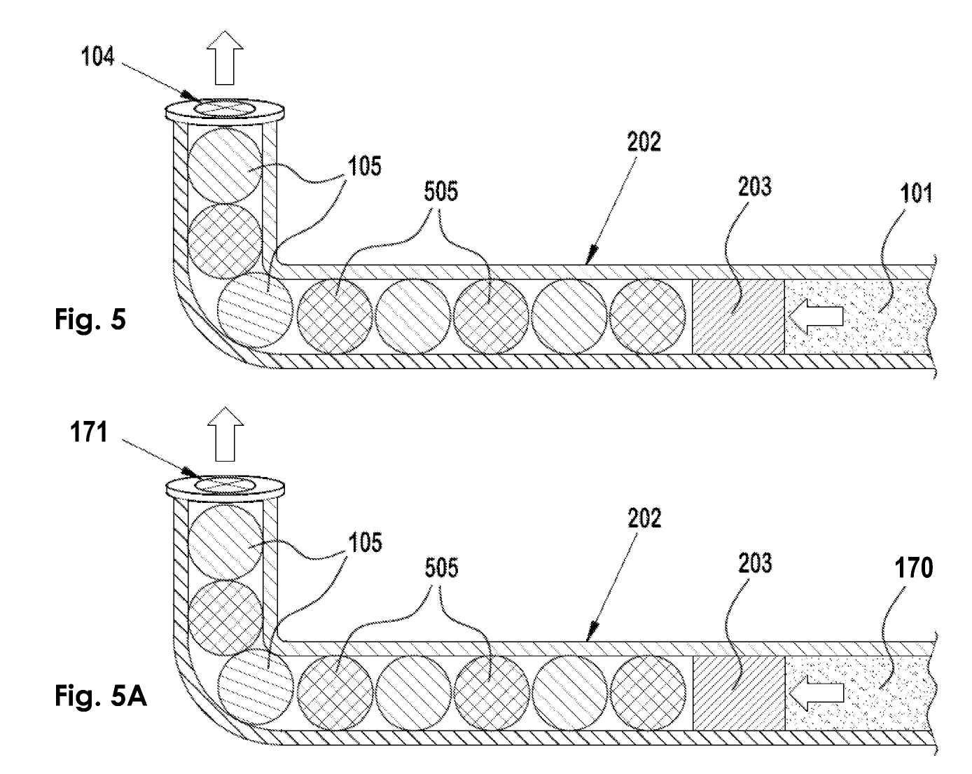

In yet another exemplary embodiment of the invention, the reservoir 100 may contain a plurality of microspheres of different composition driven through the body of the device 100 by the force of osmotic engine 101 against the cylindrical piston 203. As shown, microspheres 105 may contain a first active-agent containing material and microspheres 505 may contain a second active agent containing material (or none at all). Those skilled in the art will recognize that any number of dissimilar spheres can be used and in a variety of patterns, not simply an alternating pattern as shown illustratively in FIG. 5. In an alternate embodiment illustrated in FIG. 5A, an externally activated pumping mechanism 170 is used to propel the piston 103. The pumping mechanism 170 may be activated by an electromagnetic or radio frequency pulse or signal, magnetic, piezoelectric, electrostatic or similar means. In yet another alternate embodiment an internally generated pressure (e.g. through osmotic force) is utilized in combination with an externally actuated or activated active valve 171. Said active valve 171 may be actuated by an electromagnetic or radio frequency pulse or signal, magnetic, piezoelectric, electrostatic or similar means.

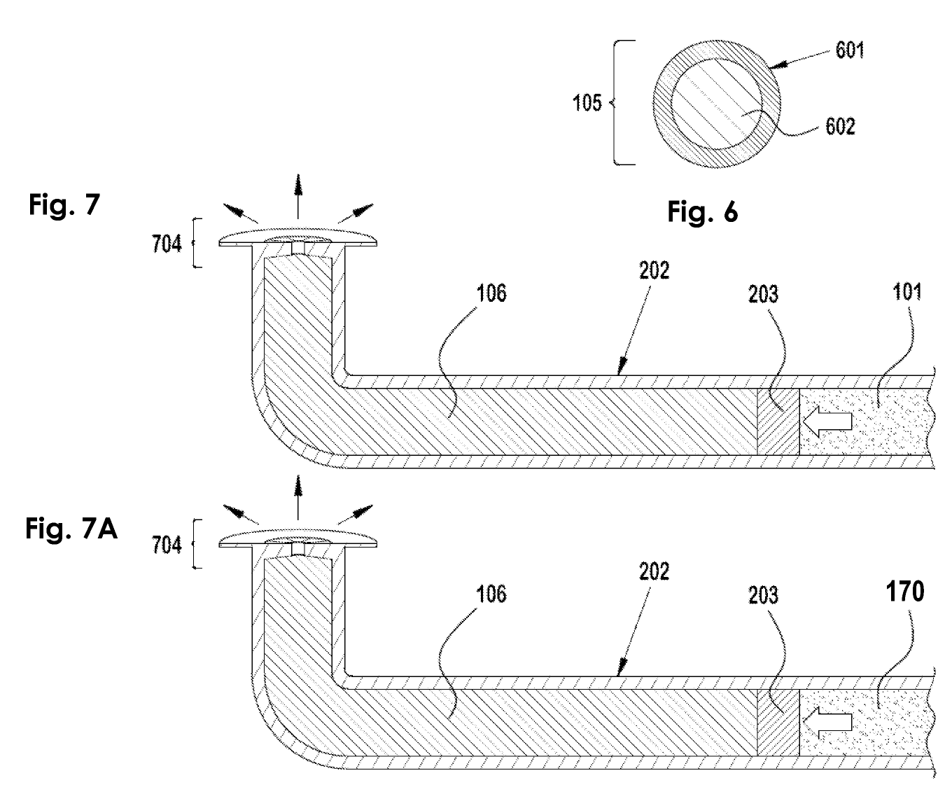

The microspheres 105, 505 may have a structure similar to that shown in FIG. 6, where microsphere 105 is shown comprising a shell coating 601 that may be generally polymeric in nature and soluble or insoluble in water; permeable or impermeable to water or active agent; biodegradable or nonbiodegradable; and rigid or elastic. The microcapsule core 602 comprises an active agent containing formulation of liquid, semisolid or solid form.

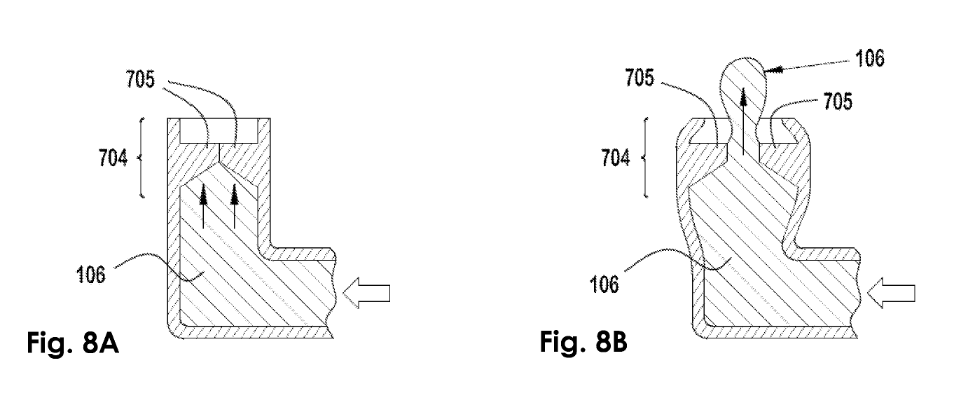

FIG. 7 illustratively shows a tubular lacrimal device 100 comprising active-agent impermeable body 202 having osmotic pump 101 disposed at a first end for exerting pressure against a piston 203. An active agent containing formulation 106 is forced through a terminal blow-off or relief valve 704 which may be configured for pressure dependent flow behavior that results in a steady osmotic pump flow being translated into a periodic pulsed or oscillatory release of active agent containing formulation, in accordance with the structure illustrated in FIGS. 8A and 8B. In an alternate embodiment illustrated in FIG. 7A, an externally activated pumping mechanism 170 is used to propel the piston 103. The pumping mechanism 170 may be activated by an electromagnetic or radio frequency pulse or signal, magnetic, piezoelectric, electrostatic or similar means.

FIGS. 8A and 8B depict of one possible embodiment of the terminal blow-off valve 704 where complementary valve elements 705 provide an elastic sealing pressure that is enhanced by an additional nonlinear magnetic, electrostatic, adhesive, capillary, or other force(s) to yield an initial valve opening pressure, aka “cracking pressure”, that significantly exceeds said sealing pressure, thereby resulting in an oscillatory or pulsed valve actuation and release of active agent containing formulation 106.

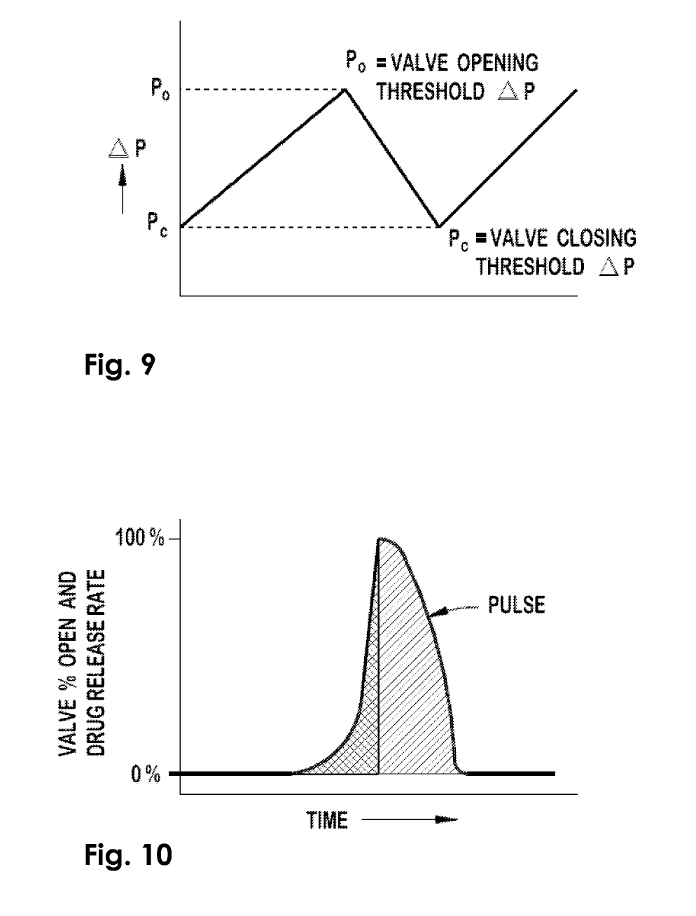

FIG. 9 shows an illustration of how the opening of valve 704 of osmotically controlled lacrimal tube device 100 of FIGS. 7 and 8 may be depicted over time. Without being bound to any specific theory, the time course of the internal pressure during one pulsed release cycle of active agent containing material may be characterized by the osmotic pump-driven pressure building to the Po valve opening pressure, the valve opening and internal pressure bleeding off as active agent containing material emits from the device, and the internal pressure falling to the valve closing pressure Pc where the valve closes. The cycle repeats itself as the steady osmotic pump begins to rebuild pressure up to Po again. One desirable range of Pc, Po is from about 20 psia to about 200 psia (where 15 psia is standard atmospheric pressure) and their difference delta-P should be large relative to ambient pressure fluctuations, i.e., greater than 1 psi and likely much greater.

FIG. 10 depicts the percent to which the valve is open and/or active agent flowrate corresponding to the pressure cycle described in FIG. 9, where valve cracking at Po is accompanied by a substantial increase in the valve opening and active agent release rate, until the pressure is bled off during the pulse and flow rate decreases to Pc, where the valve closes and substantially less active agent release is observed.

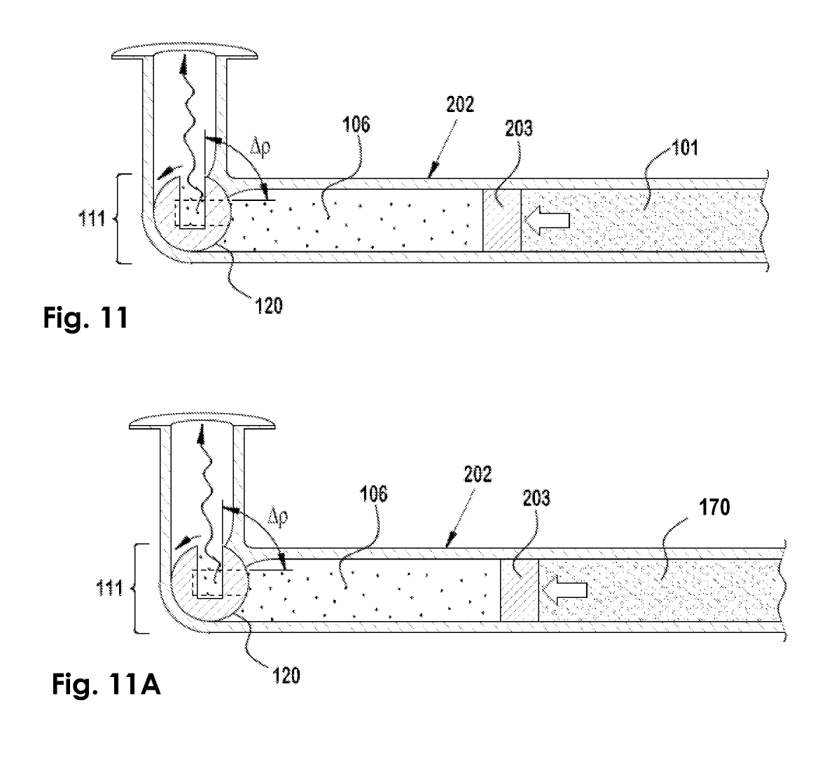

In another illustrative embodiment of the invention, FIG. 11 shows a steady osmotic pump 101 and a tubular lacrimal device body 202 (lacking stiction elements 102), driving active agent containing fluid 106 through an active or passive metering valve 120, rotary or otherwise in design, that modulates the pressure-gradient-driven flow to create pulsed or oscillatory release rates of liquid or semisolid active agent formulation. Valve stiction may be defined, for purposes of this embodiment, as the valve opening force exceeding the valve closing force. Any of valve designs known to the art can be used (ball valve, slot valve, reed valve, etc). Valve stiction can arise from mechanical interference, frictional, cohesive, capillary, or adhesive forces. Valve stiction can also arise from distance-dependent magnetic force from complementary magnetic valve elements, such as a magnetic ball and seat check valve, or distance-dependent electrostatic forces. In an alternate embodiment illustrated in FIG. 11A, an externally activated pumping mechanism 170 is used to propel the piston 103. The pumping mechanism 170 may be activated by an electromagnetic or radio frequency pulse or signal, magnetic, piezoelectric, electrostatic or similar means.

Thus, the invention may be further characterized as an osmotic or swellable hydrogel engine, with or without intermittent stops, combined with a micromechanical valving element that meters intermittent pulses of liquid or particulate drug formulation to the target site (such as the eye), via a geometrically-defined swept volume. For example, the pulses emitting from a conventional peristaltic pump, diaphragm pump, piston pump, rotating or oscillating slot valve.

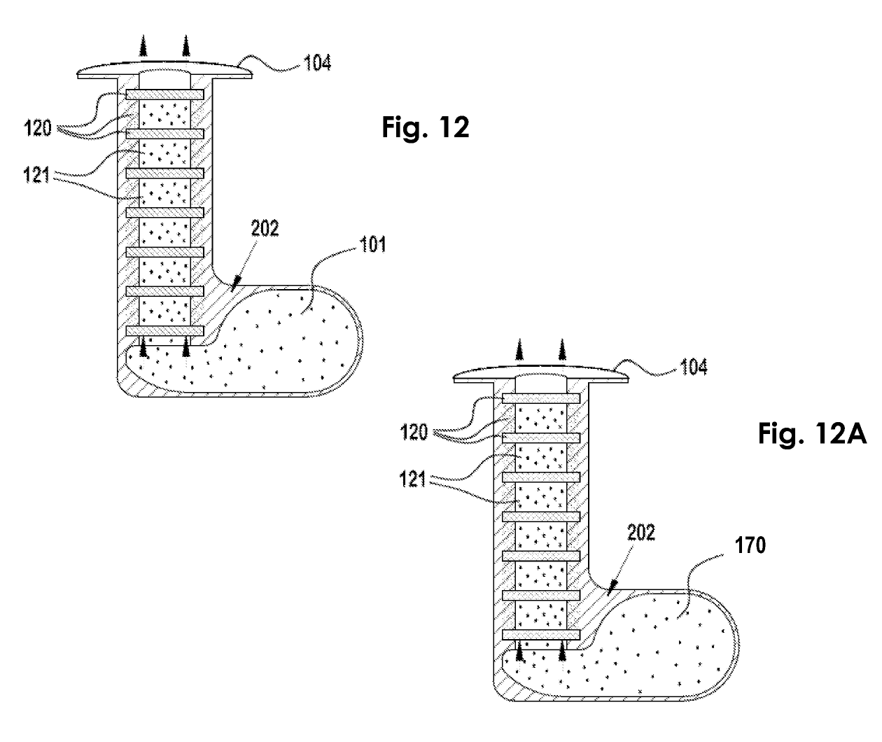

In FIG. 12, another exemplary hybrid tubular lacrimal device is shown. In this embodiment, the body 202 includes a steady osmotic pump 101 that drives a stacked series of alternating barrier layers 120 and active-agent-containing pulse layers 121. The barrier layers are active-agent-impermeable as well as non-erodible or erodible (via dissolution or biodegradation). The osmotic pump 101 pushes the entire stack of layers 120, 121 towards the terminal opening 104 of the lacrimal device 100 in order to facilitate sequential pulsed emission of the active-agent-containing layers 121. In the specific case of erodible barrier layers 120, the steady pushing of the stacked layers 120,121 towards the opening 104 of the lacrimal device 100 prevents build-up of a longer diffusion path for the active agent within the device, which otherwise progressively broadens and slows the pulsed delivery of active agent over time. In an alternate embodiment illustrated in FIG. 12A, an externally activated pumping mechanism 170 is used to propel the piston 103. The pumping mechanism 170 may be activated by an electromagnetic or radio frequency pulse or signal, magnetic, piezoelectric, electrostatic or similar means.

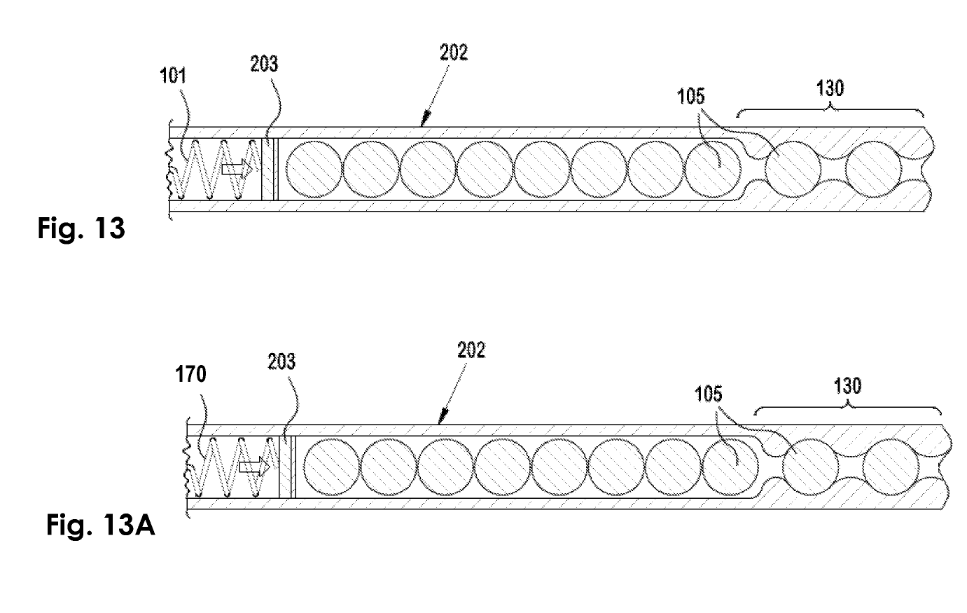

FIG. 13 illustrates another embodiment of the present invention in which a tubular lacrimal device 100, similar to those shown in FIGS. 2 and 5, comprises a steady osmotic pump 101, a body 202, and a piston 203. The piston 203 drives a plurality of active-agent-containing microspheres or microcapsules 105 sequentially through one or more complementary spherical elastic metering valve elements 130 that envelop the microspheres/capsules. The valves 130 minimize communication with the external media until the periodic emission of said microspheres/capsules 105, which are constructed to be water activated/dissolved to allow a rapid burst release of active agent. The internal interstitial fluid (not labeled) between the microspheres/capsules 105 may optionally comprise a water repelling oil to prevent premature activation of microspheres/capsules 105. In an alternate embodiment illustrated in FIG. 13A, an externally activated pumping mechanism 170 is used to propel the piston 103. The pumping mechanism 170 may be activated by an electromagnetic or radio frequency pulse or signal, magnetic, piezoelectric, electrostatic or similar means.

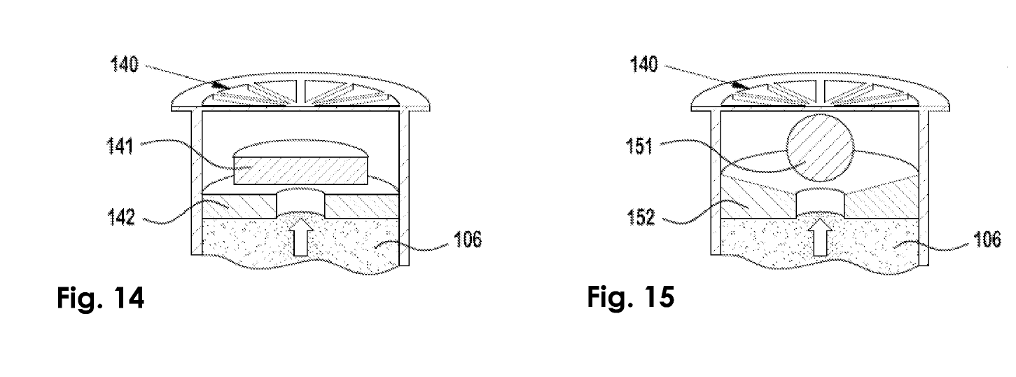

FIGS. 14-19 show various exemplary embodiments of the terminal valve 104 of FIG. 7. In FIG. 14, the valve may be comprised of a disk valve structure design comprising a porous retaining frit/grid/mesh cap that retains the disk valve element 141 but allows active agent containing formulation 106 to pass freely, and a valve seat 142. Disk valve 141 and valve seat 142 create a complementary distance dependent clamping force, such as magnetic, electrostatic, adhesive, cohesive, capillary, or other force, in order to create the valve pressure response profile similar to that depicted in FIG. 8A. Disk valve element 141 may further be connected to cap 140 via a return spring that accentuates the valve clamping force between 141 and 142. Further, as shown in FIG. 15, a porous retaining cap may be used to retain a ball valve 151 and complementary valve seat 152, wherein the valve and valve seat share a complementary distance dependent clamping force as described in FIG. 14.

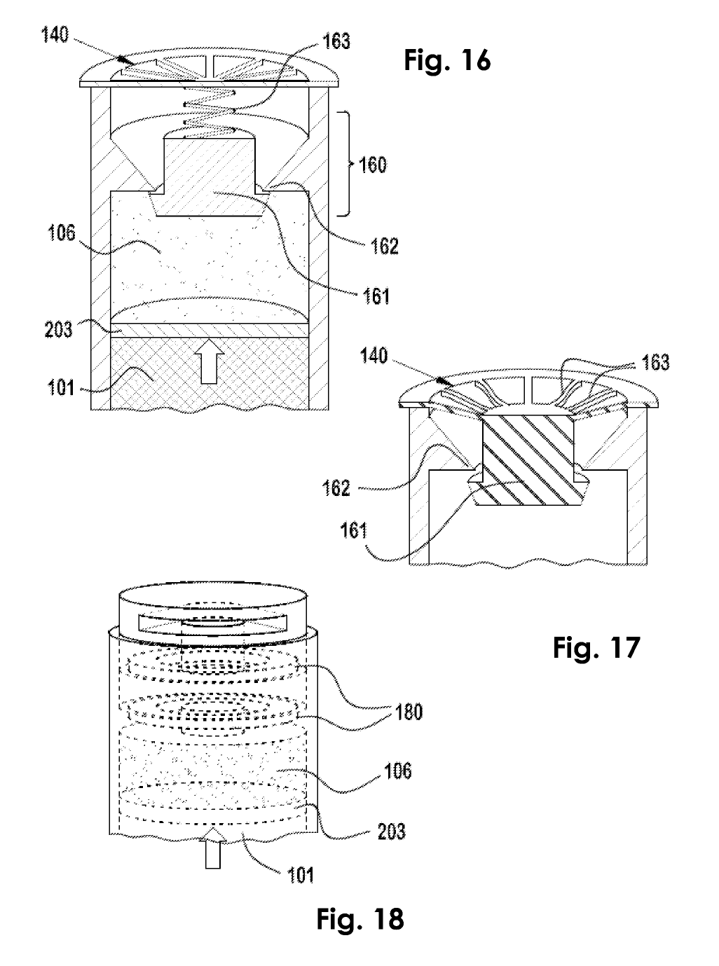

FIG. 16 depicts another embodiment of the terminal blow-off or relief valve lacrimal device depicted in FIG. 7, where a valve construct 160 comprises a porous retaining cap retains a valve plunger element 161 via a return spring 163, wherein valve plunger interacts with complementary valve seat 162 via distance dependent mechanical interference as well as optional clamping force (such as magnetic, electrostatic, adhesive, cohesive, capillary, mechanical, etc.) In FIG. 17, a return spring 163 is shown integrated into the elastic porous retaining cap.

FIG. 18 shows another embodiment of the terminal blow-off or relief valve lacrimal device depicted in FIG. 7, where the valve construct comprises a slot valve having complementary distance dependent attractive (or clamping) force surfaces 180. The clamping force may be magnetic, electrostatic, adhesion, cohesion, capillary, mechanical, or combinations thereof.

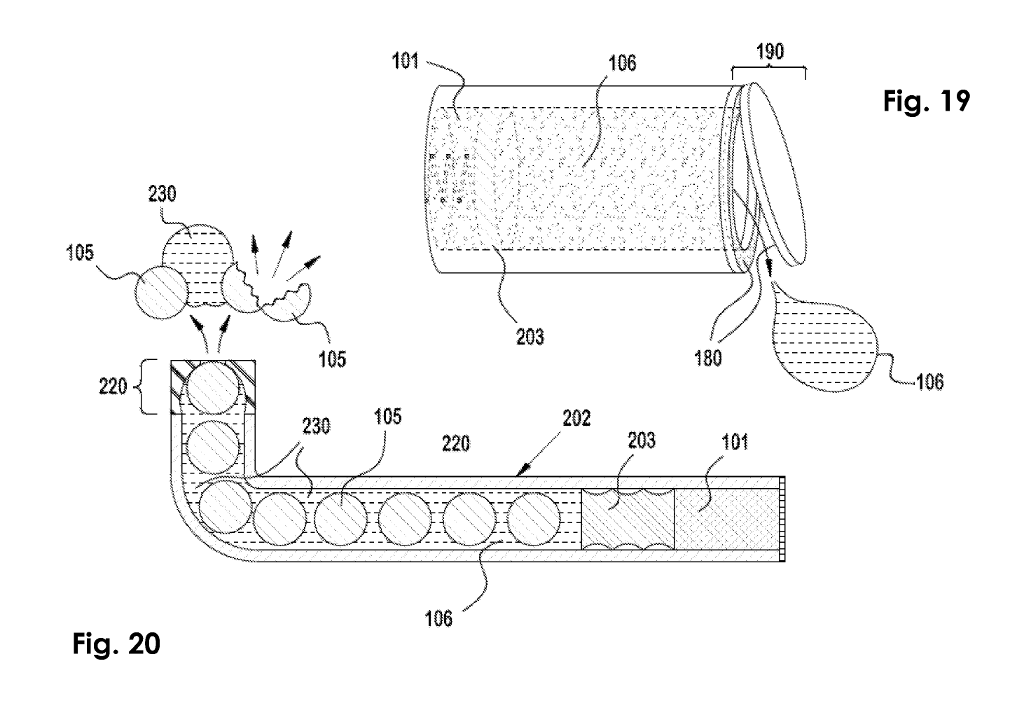

FIG. 19 illustrates another variation of FIG. 7, where a terminal flap-style blow-off or relief valve 190 is provided at the distal end of an osmotic pump device to induce an oscillatory release rate over time. The valve construct 190 comprises a flap valve having complementary distance dependent attractive or clamping force surfaces 180 that may be selected from one or more of magnetic, electrostatic, adhesion, cohesion, capillary, or mechanical in nature.

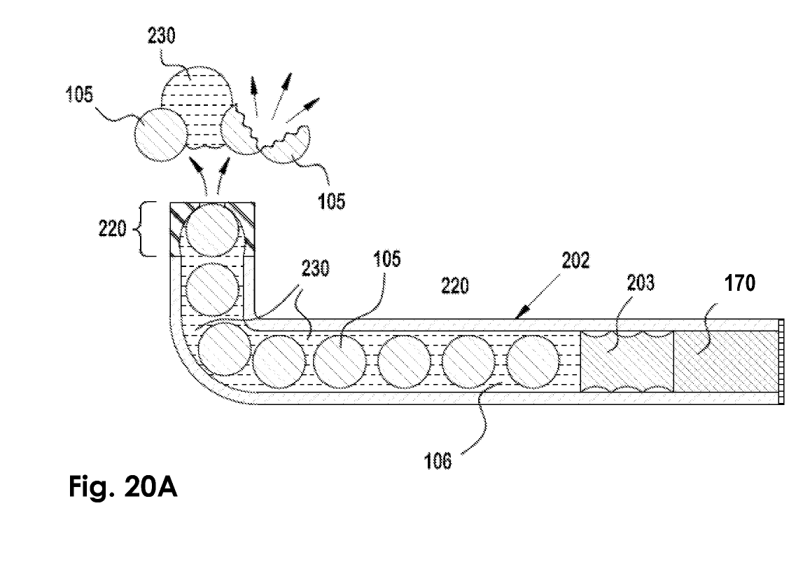

FIG. 20 shows an illustrative tubular lacrimal device 100 driven by a steady osmotic pump 101. A plurality of microspheres or microcapsules 105 may be present in reservoir 110, and a terminal restriction element 220 controls the emission of microspheres 105 from the device 100. In the case of a self-wiping elastic restriction valve 220, the interstitial fluid 230 may comprise an active-agent containing liquid or semi-solid while the microspheres 105 contain no active agent and serve as occlusive or sealing elements at the restriction valve.

When the osmotic pump 101 achieves a yield pressure sufficient to emit at least one microsphere 105, a bolus pulse of active agent containing interstitial material 230 is likewise emitted. In the case of a rigid crushing or piercing terminal element 220, where microcapsules 105 may or may not contain active agent, the osmotic pump 101 drives the microcapsules 105 to be crushed and emitted from the restriction element in sequence, also accommodated by a bolus pulse of active agent containing interstitial material 230. In an alternate embodiment illustrated in FIG. 20A, an externally activated pumping mechanism 170 is used to propel the piston 103. The pumping mechanism 170 may be activated by an electromagnetic or radio frequency pulse or signal, magnetic, piezoelectric, electrostatic or similar means.

Suitable polymeric materials for the active agent-containing material include, without limitation, hydrophobic and hydrophilic absorbable and non-absorbable polymers. Suitable hydrophobic, non-absorbable polymers include, without limitation, ethylene vinyl alcohol (“EVA”), fluorinated polymers including without limitation, polytetrafluoroethylene (“PTFE”) and polyvinylidene fluoride (“PVDF”), polypropylene, polyethylene, polyisobutylene, nylon, polyurethanes, polyacrylates and methacrylates, polyvinyl palmitate, polyvinyl stearates, polyvinyl myristate, cyanoacrylates, epoxies, silicones, copolymers thereof with hydrophobic or hydrophilic monomers, and blends thereof with hydrophilic or hydrophobic polymers and excipients.

Hydrophilic, non-absorbable polymers useful in the invention include, without limitation, cross-linked poly(ethylene glycol), poly(ethylene oxide), poly(propylene glycol), poly(vinyl alcohol), poly(hydroxyethyl acrylate or methacrylate), poly(vinylpyrrolidone), polyacrylic acid, poly(ethyloxazoline), and poly(dimethyl acrylamide), copolymers thereof with hydrophobic or hydrophilic monomers, and blends thereof with hydrophilic or hydrophobic polymers and excipients.

Hydrophobic, absorbable polymers that may be used include, without limitation, aliphatic polyesters, polyesters derived from fatty acids, poly(amino acids), poly(ether-esters), poly(ester amides), polyalkylene oxalates, polyamides, poly(iminocarbonates), polycarbonates, polyorthoesteres, polyoxaesters, polyamidoesters, polyoxaesters containing amine groups, phosphoesters, poly)anhydrides), polypropylene fumarates, polyphosphazenes, and blends thereof. Examples of useful hydrophilic, absorbable polymers include, without limitation, polysaccharides and carbohydrates including, without limitation, crosslinked alginate, hyaluronic acid, dextran, pectin, hydroxyethyl cellulose, hydroxy propyl cellulose, gellan gum, guar gum, keratin sulfate, chondroitin sulfate, dermatan sulfate, proteins including, without limitation, collagen, gelatin, fibrin, albumin and ovalbumin, and phospholipids including, without limitation, phosphoryl choline derivatives and polysulfobetains.

More preferably, the active agent-containing material is a polymeric material that is polycaprolactone. Still more preferably, the material is poly(epsilon-caprolactone), and ethylene vinyl acetate of molecular weights between about 10,000 and 80,0000. About 0 to about 100 weight percent polycaprolactone and about 100 to about 0 weight percent of the ethylene vinyl acetate are used based on the total weight of the polymeric material and, preferably, about 50% each of polycaprolactone and ethylene vinyl acetate is used.

The polymeric material used is preferably greater than about 99% pure and the active agents are preferably greater than about 97% pure. One of ordinary skill in the art will recognize that in compounding, the conditions under which compounding is carried out will need to take into account the characteristics of the active agent to ensure that the active agents do not become degraded by the process. The polycaprolactone and ethylene vinyl acetate preferably are combined with the desired active agent or agents, micro-compounded, and then extruded.

In addition to or instead of active agent loading profiles, the release kinetics may be controlled via spatial gradients of the properties of degradability and drug permeability of the active agent-containing material. For example, in those cases in which drug release kinetics are dominated by the rate of material degradation, a spatial degradation in the material chemistry including, without limitation, polylactide-glycolide copolymers of differing monomer ratios, adjacent polyglycolide and polycaprolactone layers and the like, results in spatial gradients and varied release rates as the material degradation front moves through the device. By way of further example, a material may erode more slowly initially in a first, outer material and more quickly in a second, inner material to achieve phased release kinetics.

In the case of a non-degradable material that elutes the active agent solely through diffusion-dominated mechanisms, spatial gradients in the material's permeability can control release kinetics beyond what is possible with a homogeneous material. In the diffusion-dominated mechanism, the material permeability controls release kinetics and is influenced by the material's porosity as well as the active agent solubility and diffusivity. By forming an active agent-loaded layer of an outer material with a higher permeability, the active agent elution may be controlled to be more linear with less burst effect than that which is otherwise achieved with a single, homogeneous, diffusion material.

The spatial gradients in biodegradability or permeability may be combined with continuous or step-wise gradients in the active agent loading profile. For example, a punctal plug material core having an outer segment loaded with a low active agent concentration and with a relatively low active agent permeability may be adjacent to an inner material segment loaded with a high agent concentration and with a relatively high active agent permeability, which combination achieves release kinetics unobtainable with a homogeneous material ad homogeneous active agent loading. The initial burst release is reduced and the release of the last active agent content is accelerated relative to a conventional homogeneous active agent loaded device.

Phase-separated inclusions may be used to control one or both of diffusive and degradative kinetics of the active agent-containing material. For example, water soluble polymers, water soluble salts, materials with a high diffusivity for the active agent and the like may be used as destabilizing inclusion to enhance degradation or diffusion rates. When the hydrolysis front reaches an inclusion, the inclusion rapidly dissolves and increases porosity of the active agent-containing material. The inclusions may be incorporated as gradients or layers that allow additional tailoring of the release profile.

As another alternative, a percolated network of destabilizing inclusions may be used. When used in a non-biodegradable active agent-containing material, these inclusions form islands within the material that can possess high diffusivity for the active agent. Useful inclusions will have a higher diffusivity for the active agent than the active agent-containing material. Examples of such inclusions include, without limitation, propylene glycol, silicone oil, immiscible dispersed solids such as a polymer or wax and the like. As yet another example, an inclusion that acts to adsorb water, swell the active agent-containing material and increase local diffusion kinetics may be used.

As still another alternative, stabilizing inclusions that have a low active agent diffusivity are used. These inclusions act to form a barrier that slows diffusive transport of the active agent in the vicinity of the inclusion. The overall effect is a reduction of active agent permeability in a base material that is otherwise the same. Example of such inclusions include, without limitation, micro to nano-sized silicate particles dispersed through the base material of one or both of polycaprolactone and ethylenecovinylacetate homogeneously or in continuous step-wise gradients.

The present invention encompasses numerous devices for the delivery of active agents to the eye each having various features and advantages. For example, certain devices may have a body with a first end, a second end, and a lateral surface extending between the two ends. The lateral surface preferably has an outer diameter that is substantially circular in shape and, thus, the body preferably has a cylindrical shape. A portion of the lateral surface of certain of the devices preferably has an outer diameter that is greater than the outer diameter of the remainder of the lateral surface. The enlarged portion can be any size or shape, and can be present on any part of the lateral surface, in punctal plug embodiments, the enlarged portion is of a size so that it at least partially anchors the punctal plug in the lacrimal canaliculus and preferably, the enlarged portion is at one end of the plug. One ordinarily skilled in the art will recognize that any of a wide variety of shapes are possible.

The body of the punctal plugs of the invention may take any shape and size, preferably, the body is in the shape of an elongated cylinder, e.g. tubular. The body may be from about 0.5 to about 10 mm in length. The width of the body may be from about 0.2 to about 3, preferably 0.3 to about 1.5 mm, although those skilled in the art will recognize that the sizing of the device may be wholly dependent on the size of the lacrimal puncta of the patient. Thus, larger or smaller size than those specifically recited herein may be needed in situations where the device is intended for insertion in a lacrimal puncta that is substantially larger or smaller than is typical in most human patients.

Except as where otherwise specified here for use with terminal valves or other mechanism for controlling the dispensing of active-agent containing material, the size of the opening in the lacrimal insert may be from about 1 nm to about 2.5 mm and preferably about 0.15 mm to about 0.8 mm. Instead of one large opening at any one location, multiple small openings may be used. The body of the plug may be wholly or partially transparent or opaque. Optionally, the body may include a tint or pigment that makes the plug easier to see when it is placed in a punctum.

The body of the devices of the invention may be made of any suitable biocompatible material including, without limitation, silicone, silicone blends, silicone co-polymers, such as, for example, hydrophilic monomers of polyhydroxyethylmethacrylate (“pHEMA”), polyethylene glycol, polyvinylpyrrolidone, and glycerol, and silicone hydrogel polymers such as, for example, those described in U.S. Pat. Nos. 5,962,548, 6,020,445, 6,099,852, 6,367,929, and 6,822,016, incorporated herein in their entireties by reference. Other suitable biocompatible materials include, for example: polyurethane; polymethylmethacrylate; poly(ethylene glycol); poly(ethylene oxide); polypropylene glycol); poly(vinyl alcohol); poly(hydroxyethyl methacrylate); poly(vinylpyrrolidone) (“PVP”); polyacrylic acid; poly(ethyloxazoline); poly(dimethyl acrylamide); phospholipids, such as, for example, phosphoryl choline derivatives; polysulfobetains; acrylic esters, polysaccharides and carbohydrates, such as, for example, hyaluronic acid, dextran, hydroxyethyl cellulose, hydroxyl propyl cellulose, gellan gum, guar gum, heparan sulfate, chondroitin sulfate, heparin, and alginate; proteins such as, for example, gelatin, collagen, albumin, and ovalbumin; polyamino acids; fluorinated polymers, such as, for example, PTFE, PVDF, and teflon; polypropylene; polyethylene; nylon; and EVA.

The surface of the devices may be wholly or partially coated. The coating may provide one or more of lubriciousness to aid insertion, muco-adhesiveness to improve tissue compatibility, and texture to aid in anchoring the device. Examples of suitable coatings include, without limitation, gelatin, collagen, hydroxyethyl methacrylate, PVP, PEG, heparin, chondroitin sulphate, hyaluronic acid, synthetic and natural proteins, and polysaccharides, thiomers, thiolated derivatives of polyacrylic acid and chitosan, polyacrylic acid, carboxymethyl cellulose and the like and combinations thereof.

Certain embodiments of the devices of the invention have a body made of a flexible material that conforms to the shape of whatever it contacts. Optionally, in the punctal plug embodiment, there may be a collarette formed of either a less flexible material than that of the body or material that too conforms to the shape of whatever it contacts. When a punctal plug having both a flexible body and a less flexible collarette is inserted into the lacrimal canaliculus, the collarette rests on the exterior of the lacrimal punctum and the body of the punctal plug conforms to the shape of the lacrimal canaliculus. The reservoir and the body of such punctal plugs are preferably coterminous. That is, the reservoir of such punctal plugs preferably make up the entirety of the body, except for the collarette.

In embodiments in which one or both of a flexible body and collarette are used, the flexible body and flexible collarette can be made of materials that include, without limitation, nylon, polyethylene terephthalate (“PET”), polybutylene terephthalate (“PBT”), polyethylene, polyurethane, silicone, PTFE, PVDF, and polyolefins. Punctal plugs made of nylon, PET, PBT, polyethylene, PVDF, or polyolefins are typically manufactured for example and without limitation, extrusion, injection molding, or thermoforming. Punctal plugs made of latex, polyurethane, silicone, or PTFE are typically manufactured using solution-casting processes.

Processes for manufacturing the punctal plugs useful in the invention are well known. Typically, the devices are manufactured by injection molding, cast molding, transfer molding or the like. Preferably, the reservoir is filled with one or both of at least one active agent and the active agent-containing material subsequent to the manufacture of the device. Additionally, one or more excipients may be combined with the active agent alone or in combination with the polymeric material.

The amount of active agent used in the devices of the invention will depend upon the active agent or agents selected, the desired doses to be delivered via the device, the desired release rate, and the melting points of the active agent and active agent-containing material. Preferably, the amount used is a therapeutically effective amount meaning an amount effective to achieve the desired treatment, inhibitory, or prevention effect. Typically, amounts of about 0.05 to about 8,000 micrograms of active agents may be used.

In certain aspects of the invention, the reservoir can be refilled with a material after substantially all of the active agent-containing material has dissolved or degraded and the active agent is released. For example, the new active agent-containing material can be the same as, or different from, the previous polymeric material, and can contain at least one active agent that is the same as, or different from the previous active agent. Certain punctal plugs used for particular applications can preferably be refilled with a material while the punctal plugs remain inserted in the lacrimal canaliculus, while other punctal plugs are typically removed from the lacrimal canaliculus, a new material is added, and the punctal plugs are then reinserted into the lacrimal canaliculus.

After the device is filled with the active agent, the plug is sterilized by any convenient method including, without limitation, ethylene oxide, autoclaving, irradiation, and the like and combination thereof. Preferably, sterilization is carried out through gamma radiation or use of ethylene oxide.

The devices described herein can be used to deliver various active agents for the one or more of the treatment, inhibition, and prevention of numerous diseases and disorders. Each device may be used to deliver at least one active agent and can be used to deliver different types of active agents. For example, the devices can be used to deliver azelastine HCl, emadastine difumerate, epinastine HCl, ketotifen fumerate, levocabastine HCl, olopatadine HCl, pheniramine maleate, and antazoline phosphate for one or more of the treatment, inhibition, and prevention of allergies. The devices can be used to deliver mast cell stabilizers, such as, for example, cromolyn sodium, lodoxamide tromethamine, nedocromil sodium, and permirolast potassium.

The devices can be used to deliver mydriatics and cycloplegics including, without limitation, atropine sulfate, homatropine, scopolamine HBr, cyclopentolate HCl, tropicamide, and phenylephrine HCl. The devices can be used to deliver ophthalmic dyes including, without limitation, rose bengal, sissamine green, indocyanine green, fluorexon, and fluorescein.

The devices can be used to deliver corticosteroids including, without limitation, dexamethasone sodium phosphate, dexamethasone, fluoromethalone, fluoromethalone acetate, loteprednol etabonate, prednisolone acetate, prednisolone sodium phosphate, medrysone, rimexolone, and fluocinolone acetonide. The devices can be used to deliver non-steroidal anti-inflammatory agents including, without limitation, flurbiprofen sodium, suprofen, diclofenac sodium, ketorolac tromethamine, cyclosporine, rapamycin methotrexate, azathioprine, and bromocriptine.

The devices can be used to deliver anti-infective agents including, without limitation, tobramycin, moxifloxacin, ofloxacin, gatifloxacin, ciprofloxacin, gentamicin, sulfisoxazolone diolamine, sodium sulfacetamide, vancomycin, polymyxin B, amikacin, norfloxacin, levofloxacin, sulfisoxazole diolamine, sodium sulfacetamide tetracycline, doxycycline, dicloxacillin, cephalexin, amoxicillin/clavulante, ceftriaxone, cefixime, erythromycin, ofloxacin, azithromycin, gentamycin, sulfadiazine, and pyrimethamine.

The devices can be used to deliver agents for the one or more of the treatment, inhibition, and prevention of glaucoma including, without limitation, epinephrines, including, for example: dipivefrin; alpha-2 adrenergic receptors, including, for example, aproclonidine and brimonidine; betablockers including, without limitation, betaxolol, carteolol, levobunolol, metipranolol, and timolol; direct miotics, including, for example, carbachol and pilocarpine; cholinesterase inhibitors, including, without limitation, physostigmine and echothiophate; carbonic anhydrase inhibitors, including, for example, acetazolamide, brinzolamide, dorzolamide, and methazolamide; prostoglandins and prostamides including, without limitation, latanoprost, bimatoprost, uravoprost, and unoprostone cidofovir.

The devices can be used to deliver antiviral agents, including, without limitation, fomivirsen sodium, foscarnet sodium, ganciclovir sodium, valganciclovir HCl, trifluridine, acyclovir, and famciclovir. The devices can be used to deliver local anesthetics, including, without limitation, tetracaine HCl, proparacaine HCl, proparacaine HCl and fluorescein sodium, benoxinate and fluorescein sodium, and benoxnate and fluorexon disodium. The devices can be used to deliver antifungal agents, including, for example, fluconazole, flucytosine, amphotericin B, itraconazole, and ketocaonazole.

The devices used to deliver analgesics including, without limitation, acetaminophen and codeine, acetaminophen and hydrocodone, acetaminophen, ketorolac, ibuprofen, and tramadol. The devices can be used to deliver vasoconstrictors including, without limitation, ephedrine hydrochloride, naphazoline hydrochloride, phenylephrine hydrochloride, tetrahydrozoline hydrochloride, and oxymetazoline. Finally, the devices can be used to deliver vitamins, antioxidants, and nutraceuticals including, without limitation, vitamins A, D, and E, lutein, taurine, glutathione, zeaxanthin, fatty acids and the like.

The active agents delivered by the devices can be formulated to contain excipients including, without limitation, synthetic and natural polymers, including, for example, polyvinylalcohol, polyethyleneglycol, PAA (polyacrylic acid), hydroxymethyl cellulose, glycerine, hypromelos, polyvinylpyrrolidone, carbopol, propyleneglycol, hydroxypropyl guar, glucam-20, hydroxypropyl cellulose, sorbitol, dextrose, polysorbate, mannitol, dextran, modified polysaccharides and gums, phosolipids, and sulphobetains.

1. A lacrimal insert, comprising:
a body having a first end and a second end;
a surface extending between the two ends;
a reservoir contained within the body, wherein the reservoir comprises at least one opening, an active agent-containing material and an active agent;
an externally activated pumping mechanism disposed within the reservoir; and
a piston disposed between the osmotic engine and the active-agent containing material.

2. The device of claim 1, wherein the active-agent containing material comprises a plurality of discrete particles, a porous medium, or combinations thereof.

3. The device of claim 2, wherein the plurality of discrete particles each comprising one or more therapeutic agents.

4. The device of claim 1 comprising a plurality of complementary stiction elements.

5. The device of claim 4 wherein the stiction elements have a cross sectional profile selected from one or more of hemispherical, square, rectangular, elliptical, and triangular.

6. The device of claim 1 or 4 comprising a terminal valve disposed at the first or second end of the body.

7. A lacrimal insert, comprising:
a body having a first end and a second end;
a surface extending between the two ends;
a reservoir contained within the body;
a plurality of discrete, active-agent containing, self-contained doses of therapeutic agent within the reservoir;
an externally activated pumping mechanism osmotic engine disposed at the first or second end the reservoir; and
a piston disposed between the osmotic engine and the plurality of discrete, active-agent containing, self-contained doses of therapeutic agent.

8. The device of claim 7 wherein the reservoir comprises a plurality of stiction elements.

9. The device of claim 8 wherein the stiction elements have a cross sectional profile selected from one or more of hemispherical, square, rectangular, elliptical, and triangular.

10. The device of claim 9, wherein the geometry of the stiction elements is coordinated with the motion of the osmotic engine to control the rate of disbursement of discrete, active-agent containing, self-contained doses of therapeutic agent.

11. The device of claim 7 comprising a terminal valve at an end of the body opposite to the osmotic engine.

12. The device of claim 11, wherein the terminal valve selected to coordinate with the motion of the osmotic engine to control the rate of disbursement of discrete, active-agent containing, self-contained doses of therapeutic agent.
